(19) United States
(12) Patent Application Publication
(10) Pub. No.: US 2022/0125897 A1
(43) Pub. Date: Apr. 28, 2022

(54) Antithrombin-III Treatment of Traumatic Brain Injury

(71) Applicant: THE TRUSTEES OF THE UNIVERSITY OF PENNSYLVANIA, Philadelphia, PA (US)

(72) Inventors: Jose L. Pascual, Wallingford, PA (US); Mohamed Elsaadani, Baltimore, MD (US)

(21) Appl. No.: 17/512,550

(22) Filed: Oct. 27, 2021

Related U.S. Application Data
(60) Provisional application No. 63/106,021, filed on Oct. 27, 2020.

(51) Int. Cl. A61K 38/57; A61K 31/727; A61P 25/28

(52) U.S. Cl. CPC A61K 38/57; A61P 25/28; A61K 31/727

(57) ABSTRACT

The present invention is based on the discovery that a composition comprising antithrombin-III can reduce in vivo recruitment of leukocytes and can reduce local microvascular permeability in subjects following severe traumatic brain injury (TBI). In some aspects, treatment of subjects with AT-III following a TBI improves neurocognitive recovery, in particular, with respect to learning. The present disclosure relates to a composition comprising a therapeutically effective amount of antithrombin-III for treating TBI. In another aspect, the present disclosure relates to a method of treating a TBI in a subject by administering to the subject a composition comprising a therapeutically effective amount of antithrombin-III.

### BACKGROUND

Traumatic brain injury (TBI) is a principal source of mortality, and a leading etiology of morbidity after injury in the developed world. There were approximately 2.87 million TBI-related emergency department visits, acute care hospitalizations, and deaths in the United States in 2014. Despite decades of intense laboratory and beside research, effective neuroprotection after TBI remains elusive. Moreover, impacted individuals, overrepresented by the young, may require lifelong care. Better understanding of the pathophysiology of both primary and secondary brain injury may help guide development of neurotherapeutic agents.

Primary brain injury encompasses the immediate damage incurred upon the neurons, supporting glia, and cerebral blood vessels as a result of direct external blunt force trauma to the skull and brain parenchyma. This is followed by secondary injury, a phase which can last up to weeks and is primarily driven by a cascade of neuroimmune responses believed to increase the initial scope of the injury thereby worsening neurological outcome through glial cell activation, leukocyte recruitment, and upregulation of inflammatory mediators as well as excitotoxic and apoptotic processes.

There is a need in the art for a method of treating TBI by reducing secondary injury in a subject. The present invention satisfies this unmet need.

### SUMMARY OF THE INVENTION

The present invention is based on the discovery that a composition comprising antithrombin-III can reduce in vivo recruitment of leukocytes and can reduce local microvascular permeability in subjects with TBI. In some aspects, treatment of a subject with antithrombin-III (AT-III) following a TBI improves neurocognitive recovery, in particular, with respect to learning.

In some aspects, a method is provided for treating a traumatic brain injury (TBI) in a subject, wherein the method comprises administering to the subject a composition comprising a therapeutically effective amount of antithrombin-III (AT-III).

In some embodiments, the method of treating TBI in the subject comprises administering to the subject an (AT-III composition further comprising a low molecular weight heparin.

In some embodiments, the method of treating TBI in the subject comprises administering to the subject an AT-III composition further comprising a low molecular heparin, wherein the heparin is enoxaparin.

In some embodiments, the method of treating TBI in the subject comprises administering to the subject a therapeutically effective amount of AT-III, or a therapeutically effective amount of AT-III with a low molecular heparin, wherein the composition is administered to the subject intravenously or subcutaneously.

In some embodiments, the method of treating TBI in the subject according to any of the above embodiments reduces secondary injury in the subject following the TBI by blocking neurovascular inflammation, reducing cerebral endothelial-leukocyte interactions, decreasing blood brain barrier permeability, or a combination thereof.

In some embodiments, the method of treating TBI in the subject according to any of the above embodiments improves post-TBI cognitive recovery in the subject.

In some embodiments, the method of treating a TBI in the subject according to any of the above embodiments improves post-TBI learning and memory in the subject.

In some embodiments, the method of treating TBI in the subject according to any of the above embodiments comprises the step of administering to the subject a composition comprising a therapeutically effective amount of AT-III, wherein the composition is administered within 72 hours following the TBI.

In some embodiments, the method of treating TBI in the subject comprises administering a therapeutically effective amount of AT-III within one hour following the TBI.

In some aspects, the invention provides a composition for treating a traumatic brain injury (TBI) in a subject, the composition comprising a therapeutically effective amount of AT-III.

In some embodiments, the composition for treating TBI in the subject comprises a therapeutically effective amount of an AT-III and further comprises a low molecular weight heparin.

In some embodiments, the composition for treating TBI in the subject comprises an AT-III composition further comprising a low molecular heparin, wherein the heparin is enoxaparin.

In some embodiments, the composition for treating TBI according the any of the above embodiments is administered to the subject intravenously or subcutaneously.

In some embodiments, the composition for treating TBI in the subject according to any of the above embodiments reduces secondary injury in the subject following the TBI by blocking neurovascular inflammation, reducing cerebral endothelial-leukocyte interactions, decreasing blood brain barrier permeability, or a combination thereof.

In some embodiments, the composition for treating TBI in the subject according to any of the above embodiments improves post-TBI cognitive recovery in the subject.

In some embodiments, the composition for treating a TBI in the subject according to any of the above embodiments improves post-TBI learning and memory in the subject.

In some embodiments, the composition for treating TBI in the subject according to any of the above embodiments comprises a therapeutically effective amount of AT-III, wherein the composition is administered within 72 hours following the TBI.

In some embodiments, the composition for treating TBI in the subject comprises a therapeutically effective amount of AT-III within one hour following the TBI.

In some embodiments, the composition for treating TBI in the subject further comprises a pharmaceutically acceptable carrier.

The skilled artisan will understand that the AT-III composition disclosed herein can be administered to a subject alone or in combination with additional treatments known or believed to reduce secondary injury and/or improve patient outcomes in a subject following a TBI.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIG. 1 depicts a timeline of the experimental procedures. CCI: Controlled Cortical Impact, AT-III: antithrombin III, ENX: Enoxaparin, NS: Normal Saline, wt: Weight.

FIGS. 2A-2B depict in vivo leukocyte/endothelial interactions. FIG. 2A: A representative image showing in vivo LEUs interacting with pial microvascular endothelium. White arrows indicate fluorescently labeled LEU transiting on the endothelium. FIG. 2B: Leukocyte transit and adherence in the pial penumbral microcirculation 48 hours after CCI. Compared with untreated injured animals (CCI+VEH), both AT-III and ENX reduced post-TBI LEU transit and adherence. (*p<0.05, **p<0.01 vs. CCI+VEH).

FIGS. 3A-3B depict in vivo cerebral microvascular permeability. FIG. 3A: A representative image of FITC-albumin leakage in the cerebral microcirculation. The microvascular permeability index equals the ratio of mean fluorescence of three separate locations outside the vessel wall (perivenular intensity, IP) to mean fluorescence of three separate locations within venule (venular intensity, IV). FIG. 3B: FITC-albumin leakage in the pial penumbral microcirculation 48 hours after CCI. Compared with untreated injured animals (CCI+VEH), only AT-III reduced post-TBI cerebrovascular albumin leakage (*p<0.05 vs CCI+VEH). The boxplot contains the median (−) and the mean (×).

FIGS. 4A-4B depict body weight and Garcia Neurological Test Scores. FIG. 4A: Body Weight Assessment: All the groups scored similarly at 24 h, but at 48 h AT-III treatment significantly reduced weight loss as compared to CCI+VEH (*p<0.01 vs. CCI+VEH for given time frame). The mean initial body weight of each group was as follows: SHAM+VEH=33.4+/−0.6 g, SHAM+ENX=34.2+/−0.6 g, SHAM+ATIII=32.1+/−1.2 g, CCI+VEH=31.5+/−0.8 g, CCI+ENX=33.0+/−0.5 g, CCI+ATIII=30.9+/−1.3 g, wherein SHAM refers to sham craniotomy. FIG. 4B: Garcia Neurological Test score: all uninjured animals scored significantly better than injured counterparts (*p<0.01 vs. CCI+VEH for given time frame). Neither AT-III nor ENX treatment injured groups were significantly different from the injured untreated group.

FIG. 5 depicts the timeline for the second set of experimental procedures. CCI: Controlled Cortical Impact, AT-III: antithrombin III.

FIGS. 6A-6B depict a comparison of cued cumulative learning in uninjured (SHAM) mice, injured untreated mice (CCI+VEH), and injured mice treated with AT-III (CCI+AT-III). FIG. 6A, time to reach platform at day 6, day 7 and day 8 post TBI; FIG. 6B, time to reach platform (mean cumulative).

FIGS. 7A-7B depict a comparison of spatial learning in uninjured (SHAM) mice, injured untreated mice (CCI+VEH), and injured mice treated with AT-III (CCI+AT-III). FIG. 7A, time to reach platform at day 9, day 10, day 11, day 12 and day 13 post TBI; FIG. 7B, time to reach platform (mean cumulative).

FIGS. 8A-8B depict probe memory trails in uninjured (SHAM) mice, injured untreated mice (CCI+VEH), and injured mice treated with AT-III (CCI+AT-III). FIG. 8A, time to reach platform at day 10, day 11, day 12, day 13 and day 14 post TBI; FIG. 8B, time to reach platform (mean cumulative).

### DETAILED DESCRIPTION OF THE DISCLOSURE

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, selected methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “composition” or “pharmaceutical composition” refers to at least one compound useful within the invention that is optionally mixed with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject, or individual is a human.

As used herein, the term “treatment” or “treating” is defined as the application or administration of a therapeutic agent, i.e., a compound of the disclosure (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

As used herein, AT-III refers to a small glycoprotein anticoagulant that inactivates several enzymes of the coagulation system. It accounts for most of the antithrombin activity in plasma and also inhibits other proteolytic enzymes. It circulates in the plasma and inactivates thrombin. AT-III inhibits clotting factors IIa (thrombin), Xa, and to a lesser extent IXa and XIIa. AT-III is an α2-globulin with a mass of about 5 to 8 kDa and comprises about 425 amino acids. It has a high degree of sequence similarity with α1

-antitrypsin. Two AT-III isoforms occur naturally in human plasma. The α-ATIII isoform has four N-glycans attached to Asn 96, 135, 155, and 192. The β-ATIII isoform lacks carbohydrate on Asn 135 (N135), which is near the heparin binding site, and binds heparin with higher affinity than does α-ATIII.

As used herein, secondary injury refers to the pathophysiological occurrences in the hours and days that follow TBI. Mechanisms underlying the impact of secondary injury are known to include inflammatory, excitotoxic, and apoptotic processes, with neuroinflammatory events that activate glial cells and trafficking neutrophils, and upregulation of both local and regional inflammatory mediators.

### Compositions

In one aspect, the present invention relates to a composition comprising a serine protease inhibitor (serpin). The In one embodiment, the serpin is an anticoagulant. In one embodiment, the serpin is antithrombin. In some embodiments, the antithrombin is AT-III.

In one embodiment, the composition further comprises a heparin. In some embodiments, the heparin is a low molecular weight heparin. The low molecular weight heparin can be any low molecular weight heparin known to a person of skill in the art. Exemplary low molecular weight heparins include, but are not limited to, bemiparin, nadroparin, reviparin, enoxaparin, certoparin, dalteparin, tinzaparin, and combinations thereof. In some embodiments, the heparin is enoxaparin (ENX).

In one embodiment, the composition comprises a solvent. The solvent can be any solvent known to a skilled artisan to be safe for administration to a mammal. In one embodiment, the solvent is an aqueous solvent. Exemplary aqueous solvents include, but are not limited to, tap water, distilled water, deionized water, saline, sterile water, filtered water, and combinations thereof. In some embodiments, the solvent is normal saline. In other embodiments, the solvent is sterile water.

In some embodiments, the composition comprises lyophilized AT-III with a potency of between about 50 IU and about 5,000 IU which is reconstituted in about 1 mL to about 100 mL of solvent. In some embodiments, the composition comprises lyophilized AT-III with a potency of about 450 IU and about 550 IU which is reconstituted in about 5 mL to about 15 mL of solvent. In some embodiments, the composition comprises lyophilized AT-III with a potency of about 500 IU reconstituted in about 10 mL of solvent. In some embodiments, the composition comprises lyophilized AT-III with a potency of about 500 IU reconstituted in about 10 mL of sterile water.

In certain embodiments, the composition comprises an inactive ingredient. The inactive ingredient may be any inactive ingredient known to a person of skill in the art. In certain embodiments, the inactive ingredient is selected from the group consisting of excipients, diluents, fillers, binders, disintegrants, lubricants, colorants, preservatives, stabilizers, viscosity increasing agents, sweeteners, flavoring agents, and any combinations thereof.

### Methods

In another aspect, the present invention relates to a method of treating a traumatic brain injury in a subject, the method comprising: administering to the subject a composition comprising a therapeutically effective amount of antithrombin-III.

The composition comprising AT-III can be administered to the subject using any method known to a person of skill in the art. In one embodiment, the composition is administered intravenously. In another embodiment, the composition is administered subcutaneously. The composition can comprise any additional active or inactive ingredients known to a person of skill in the art. In one embodiment, the composition further comprises a heparin. Exemplary heparins are described elsewhere herein. In some embodiments, the heparin is ENX. In some embodiments, the composition further comprises a solvent. Exemplary solvents are described elsewhere herein.

The composition can comprise any concentration of AT-III known to a skilled artisan to be safe for administration to a mammal and to provide the desired therapeutic effect. In some embodiments, the composition comprises between about 5 mg and about 100 mg of a heparin. In some embodiments, composition comprises between about 25 mg to about 50 mg of a heparin. In other embodiments, the composition comprising AT-III and a heparin is administered to the subject to provide a dosage of heparin that is between about 0.25 mg/kg and about 5 mg/kg. In some embodiments, the dosage of heparin is between about 0.75 mg/kg and about 2.0 mg/kg. In some embodiments, the above dosages of heparin are administered daily over the course of multiple days. Although not wishing to be limited by theory, compositions comprising both AT-III and a heparin may have an added anti-coagulability effect, resulting in bleeding in the brain (hemorrhage expansion). Therefore, in embodiments wherein the composition comprises AT-III and a heparin, the amounts of AT-III and heparin in the composition will be determined such that they provide the beneficial effects of reducing inflammation and/or swelling of brain tissue in the subject without causing adverse effects such as bleeding in the brain.

The composition can be administered to the subject any time post-TBI. In one embodiment, the composition is administered to the subject as close as possible after the TBI has occurred. In one embodiment, the composition is administered to the subject within an hour following the TBI. In another embodiment, the composition is administered to the subject within 24 hours, within 48 hours, or within 72 hours following the TBI. In some embodiments, the composition comprising AT-III is administered to the subject more than once.

In some embodiments, the step of administering a composition comprising AT-III to the subject provides treatment for a TBI by reducing secondary brain injury following TBI. In some embodiments, the composition comprising AT-III reduces secondary injury by blocking neurovascular inflammation, reducing cerebral endothelial-leukocyte interactions, decreasing blood brain barrier permeability, or a combination thereof. Although not wishing to be limited by theory, it is believed that AT-III acts a potent anti-inflammatory agent and further acts to reduce leukocyte (LEU) activation. The anti-inflammatory effect of AT-III may be mediated through interactions between AT-III and endothelium, producing a profound increase in endothelial prostacyclin production, which blocks LEU adhesion to endothelial cells (ECs). In some embodiments, the composition comprising AT-III improves post-TBI cognitive recovery (e.g. learning and/or memory) in the subject.

In some embodiments, the method further comprises the step of administering to the subject an additional TBI treatment. The additional TBI treatment can be any TBI treatment known to a person of skill in the art to reduce secondary injury and/or improve patient outcomes following a TBI. Exemplary additional TBI treatments include, but are not limited to, surgery, medication (e.g. pain relievers, anti-anxiety medications, anticoagulants, anticonvulsants, antidepressants, muscle relaxants, stimulants), rehabilitation therapy (e.g. physical therapy, occupational therapy, speech therapy, psychological counseling, vocational counseling, cognitive therapy), and combinations thereof. The additional TBI treatment can be administered before, after, or concurrent with the composition comprising AT-III.

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Antithrombin-III Ameliorates Post-TBI Cerebral Leukocyte Mobilization Enhancing Recovery of Blood Brain Barrier Integrity

### Materials and Methods

### Experimental Design and Study Groups:

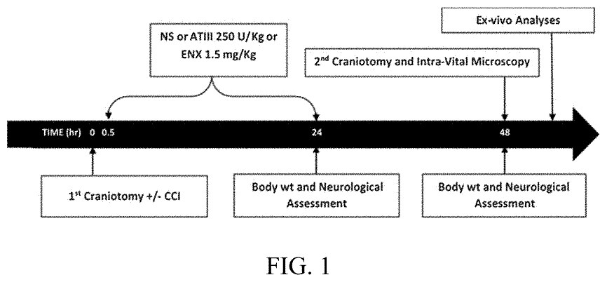

Procedures received approval from the University of Pennsylvania Institutional Animal Care and Use Committee. CD1 adult male mice (25-30 g) (Charles River Laboratories; Wilmington, Mass.) were acclimated in standard housing with ad libitum water and chow for 5 days before experiments and immediately after recovery from brain injury. Mice underwent controlled cortical impact (CCI) or sham craniotomy (SHAM) and then received one of three agents: 1) subcutaneous enoxaparin (ENX, 1.5 mg/kg) (Winthrop, Sanofi-Aventis; Bridgewater, N.J.); intravenous (femoral vein) pharmaceutical grade antithrombin-III (AT-III, 250 IU/kg, donated by Grifols S.A. (Durham, N.C.); or 3) an equal volume of IV normal saline (0.9% NS, Baxter; Deerfield, Ill.). Doses were administered 0.5 and 24 hours after the CCI (FIG. 1). AT-III dosing was chosen based on discussions with the manufacturer scientific board and previous published reports while enoxaparin doses were equivalent to therapeutic doses in humans and reflected previous literature demonstrating beneficial effects in reducing brain injury after TBI (Li, S. et al., J. Trauma Acute Care Surg., 2015, 79:78-84).

Seventy-one (71) mice were randomly assigned to one of six groups: 1) sham craniotomy and vehicle (VH, 0.9% normal saline, Baxter Healthcare Corporation; Deerfield, Ill.), (SHAM +VEH, n=9); 2) sham craniotomy and ENX (SHAM+ENX, n=8); 3) sham craniotomy and AT-III (SHAM+AT-III, n=9); 4) TBI and vehicle (CCI+VEH, n=19); TBI and ENX (CCI+ENX, n=13); and 6) TBI and AT-III (CCI+AT-III, n=13).

### Severe TBI Model:

CCI was used to replicate TBI through a well-validated rodent model (16, 17). Briefly, on day 1, mice were anesthetized with intraperitoneal ketamine (Hospira; Lake Forest, Ill.), xylazine (Alcorn; Decatur, Ill.), and acepromazine (Boehringer Ingelheim; St. Joseph, Mo.) (KXA: 100, 10, 1 mg/kg, respectively) and place prone in a stereotactic device. After scalp exposure, a left-sided 4-mm craniotomy was created between bregma and lambda structures using a dental drill (Henry Schein; Melville, N.Y.) without violating the dura. The left parietotemporal cortex was then injured via a controlled cortical impactor (CCI) (AMS201, AmScien Instruments; Richmond, Va.) which resulted in a reproducible injury previously correlated to severe TBI (3-mm-diameter impactor tip, impact velocity of 6 m/s, and cortical deformation depth of 1 mm).

### Pial Intravital Microscopy:

In vivo assessment of cerebral microcirculation using pial intravital microscopy was performed 48 hours after CCI as described previously, as this is the optimal time to observe peak leukocyte mobilization to the BBB penumbra after brain injury. Under KXA anesthesia, the right jugular vein was cannulated and a second 2.5 mm craniotomy was created immediately anterior to the first and covered with a 5 mm coverslip (Fisher Scientific; Waltham, Mass.). Secured in the stereotactic device, mice were transferred to an intravital microscope (ECLIPSE, FN1, Nikon Instruments; Melville, N.Y.) and received a 50 μL intrajugular injection of 0.3% rhodamine 6G (Sigma-Aldrich; St. Louis, Mo.) to fluorescently label circulating LEUs subsequently visualized at a 590 nm epi illumination emission exposure. A randomly selected area of non-branching pial venules measuring 25-50 μm in diameter was selected for a 1 minute digital video recording (QuantEM camera, Photometrics; Tucson, Ariz.). Intravenous bovine fluorescein isothiocyanate (FITC) labelled albumin (100 mg/kg) (Sigma-Aldrich; St. Louis, Mo.) was then administered to visualize albumin leakage in the same venule as a surrogate for microvascular permeability. Permeability was observed under a 488 nm fluorescent filter. A 10 second digital video recording was captured for offline assessment of microvascular permeability.

### Offline Quantification of LEU/EC Interactions and Albumin Leakage:

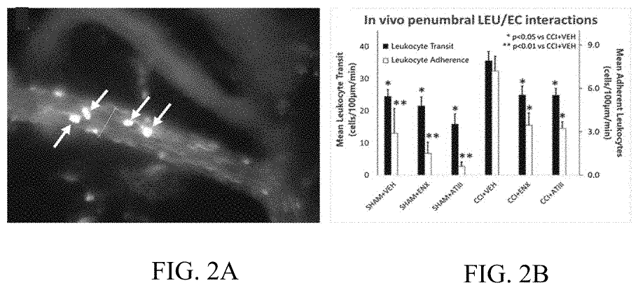

Video data captured at 590 nm λ were imported into analysis software (NIS-Elements, Nikon Instruments; Melville, N.Y.) and LEU/EC interactions were counted by a blinded observer quantifying the following parameters: 1) LEU transit: mean number of labelled EU crossing a 100 μm long venular segment in 60 seconds; and 2) LEU adhesion: number of LEU stationary for at least 30 seconds during the recording period. Fluorescently labelled spherical cells measuring 7 to 12 μm were counted as LEU and interactions were reported as the number of cells/100 μm per minute (FIGS. 2A and 2B).

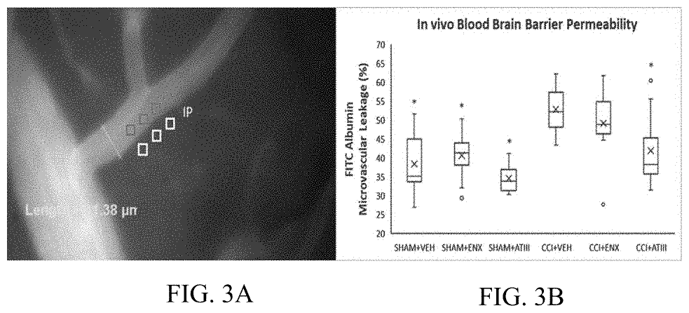

Footage captured under the 488 nm filter was evaluated for fluorescence intensity from FITC-labelled albumin measured in three distinct regions within the vessel (venular intensity) and outside the vessel walls (perivenular intensity) (FIGS. 3A and 3B). The ratio of mean venular intensity to mean perivenular intensity was averaged to determine the permeability index for a given vessel indicating the degree of macromolecular vessel leakage.

### Body Weight Loss, Garcia Neurological Test:

Animal body weights (BW) were obtained just before (W0h) as well as 24 and 48 hours after CCI with animal weight loss expressed as a ratio: (W0h-W24h or W48h)/W0h×100%. Murine neurologic function was assessed by the modified Stroke Garcia Neurologic Test (GNT) assessing motor, sensory, reflex, and balance ability at 24 hours and 48 hours after TBI. 18 points is the maximum achievable score.

### Brain Water Content:

Forty-eight hours after CCI, mice were sacrificed and excised brains were separated into injured (ipsilateral) and uninjured (contralateral) hemispheres. Wet weight (WW) was immediately assessed. Dry weight (DW) was assessed after 72 hours of dehydration at 70° C. Tissue water content was calculated using wet-to-dry ratios: % water content=100×(WW−DW)/WW.

### Statistical Analysis:

All data are presented as mean or median (as marked)±SEM. Statistical analyses were performed using SPSS (SPSS; Chicago, Ill., 2019). Differences between group means were compared using the Kruskal-Wallis test; significance assumed for p<0.05.

### Results

### In Vivo LEU-EC Interactions and Microvascular Permeability (48 Hours):

Forty-eight hours after CCI, injured animals demonstrated great number of LEC/EC interactions and microvascular leakage; uninjured control mice demonstrated the lowest levels of both parameters. LEU transit in CCI+VEH mice (35.6±2.9 LEUs/100 um/min) was reduced by administration of both ENX (24.9±2.8 LEUs/100 um/min, p=0.016) and AT-III (24.8±2.1 LEUs/100 um/min, p=0.026) (FIGS. 2A and 2B). Both treatments (ENX, AT-III) resulted in similar LEU transit levels. Specifically, LEU adhesion in CCI+VEH mice (7.2±1 LEUs/100 um/min) was reduced by ENX (3.5±0.8 LEUs/100 um/min, p=0.03) and AT-III (3.2±0.5 LEUs/100 um/min, p=0.048).

Cerebrovascular FITC-albumin leakage (FIGS. 3A and 3B) was greater in CCI+VEH (52.8±1.3%) compared to CCI+AT-III mice (41.9±2.8%, p=0.002), but similar to CCI+ENX mice (49.1±2.3%, p>0.05).

### Brain Edema (48 Hours):

Uninjured animals receiving no treatment demonstrated less ipsilateral cerebral hemisphere edema compared to injured counterparts (Table 1). Treatment with AT-III or with ENX did not significantly reduce cerebral edema as compared to CCI+VEH (p>0.05). Contralateral cerebral hemisphere edema was similar in all groups (p>0.05).

TABLE 1
Brain edema in treated animals compared to untreated animals

| | SHAM + VEH | SHAM + ENX | SHAM + ATIII | CCI + VEH | CCI + ENX | CCI + ATIII |
|---|---|---|---|---|---|---|
| Ipsilateral | 76.64 | 77.4716 | 77.66745 | 78.51845 | 78.3674 | 77.64315 |
| (%) Mean | | | | | | |
| Ipsilateral | 77.66 | 77.69902 | 77.68606 | 78.68589 | 79.02885 | 78.6192 |
| (%) Median | | | | | | |
| Ipsilateral | 0.74 | 0.184223 | 0.224232 | 0.215833 | 0.546356 | 1.10583 |
| SEM | | | | | | |
| Contralateral | 76.65 | 77.5164 | 77.77869 | 77.44365 | 77.26 | 75.11578 |
| (%) Mean | | | | | | |
| Contralateral | 77.47 | 77.67654 | 77.83964 | 77.56415 | 77.37609 | 77.69782 |
| (%) Median | | | | | | |
| Contralateral | 0.65 | 0.173227 | 0.195306 | 0.184909 | 0.403147 | 2.104034 |
| SEM | | | | | | |
| Ratio | 0.999 | 0.999 | 0.998 | 1.014 | 1.014 | 1.033 |
| (ipsi/contra) | | | | | | |

### Body Weight Loss, Garcia Neurological Test (24 and 48 Hours):

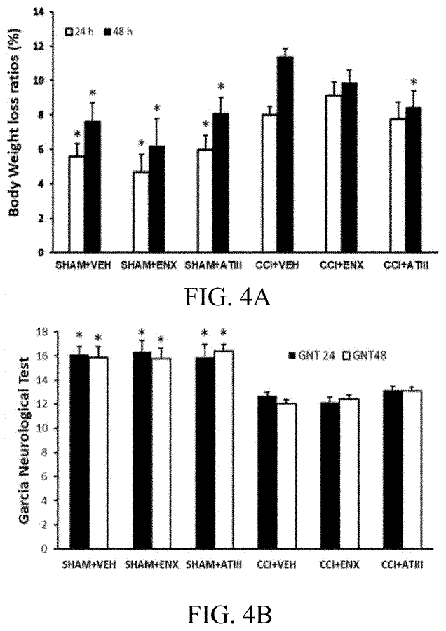

Body weight loss was greatest 48 hours after TBI, particularly in injured untreated animals (CCI+VEH, 11.4±0.5%) (FIG. 4A). Among injured animals, only AT-III treatment significantly reduced (8.4±1%, p<0.01) the weight loss observed in CCI+VEH animals. All uninjured groups (SHAM) also demonstrated less body weight loss than CCI+VEH animals (p<0.01).

The stroke functional recovery score (GNT) was highest in uninjured animals compared to all other groups (FIG. 4B). All injured mice demonstrated similar 24 or 48 hour post-TBI GNT scores (untreated 12.0±0.3; ENX 12.4±0.4; AT-III 13.1±0.3, p>0.05), with the highest scores in the AT-III treated mice, but failing to reach significance.

### Selected Discussion

In the present disclosure, in vivo evidence is provided demonstrating the ability of AT-III to reduce leukocyte activation in the injured peri-contusional neurovasculature, blocking its interactions with the blood brain barrier. The data further demonstrations how, in the same pial vessels, AT-III preserves blood brain barrier integrity, decreasing macromolecular leakage by more than 25%. AT-III animals appear to derive some clinical benefit, recovering lost weight earlier.

The economic and human burden of TBI is enormous, yet few interventions discovered in the last decades have led to significant curbing of TBI-related mortality. Key to finding a beneficial therapy is the greater understanding of the underpinnings of brain injury pathophysiology. TBI can be described as having two separate, but interconnected components. The first component is a primary injury, which occurs upon external impact to the skull, immediately causing mechanical damage to neurons, axons, glia, and cerebral blood vessels. An early occurrence after primary injury is the disruption of the blood brain barrier, enabling interchange between the plasma space and the injured cerebral parenchyma. The second component is a secondary injury, which lasts for subsequent hours and days and which is primarily driven by the host response to tissue injury. The secondary injury is characterized by an internal subsequent cascade of neuroimmune mechanisms believed to cause further tissue damage which can, in some cases, ultimately lead to brain herniation and death.

Local penumbral activation of the innate immune response, specifically circulating LEU and neurovascular endothelium, is believed to initiate and sustain the initial BBB disruption, increasing microvascular permeability and leading to worsening cerebral edema (Lukaszewicz, A. C. et al., Curr. Opin. Anaesthiol., 2011, 24:138-143; von Leden, R. E. et al., Exp. Neurol., 2019, 317:144-145). Locally curated LEUs release inflammatory mediators that further mobilize glia and other immune cells to the site of the injury and aggravate neuroinflammation (Morganti-Kossmann, M. C. et al., Shock, 2001, 16:165-177; Kumar, A. et al., Brain Behay. Immun., 2012, 26:1191-1201). The influx of plasma and blood components combined with cytotoxic cell membrane dysfunction exacerbates local edema, increases intracranial pressure, and promotes local and global cerebral ischemia (Morganti-Kossmann, M. C. et al., Acta Neuropathol. (Berl.), 2019, 137: 731-755). Activated circulating leukocytes marginate out of laminar flow, interacting with and adhering to concurrently activated ECs and transmigrating through the microvasculature into the brain parenchyma (Zhang, Z. et al., Neuroscience, 2006, 141:637-644). These leukocytes are mostly polymorphonuclear neutrophils (PMNs) but also include macrophages and T lymphocytes (DiStasti, M. R. Trends Immunol., 2009, 30:547-556). Local PMN accumulation increases in the first 24 hours after injury and peaks at 48-72 hours when critical loss of BBB integrity is observed (Morganti-Kossmann, M. C. Acta Neuropathol. (Berl), 2019, 137:731-755). Specifically, peripheral LEU infiltration into brain tissue activates resident immune cells, such as microglia and astrocytes, which has between linked to an imbalance between local concentrations of proinflammatory and anti-inflammatory cytokines (Lucas, S. M. et al., Br. J. Pharmacol., 2006, 147:232-240; Kubes, P. et al., Brain Pathol., 2006, 5:127-135). Locally activated leukocytes release metalloproteinases (MMPs), proteases, reactive oxygen species (ROS), tumor necrosis factor α (TNF-α), interleukin-1β (IL-1 β), and interleukin-6 (IL-6) (DiStasi, M. R. et al., Trends Immunol., 2009, 30:547-556) within hours from injury, further aggravating BBB disruption (Nguyen, H. X. et al., J. Neurochem., 2007, 102:900-912; Werner, C. et al., Br. J. Anaesth., 2007, 99:4-9). Tissue leukocyte infiltration is further facilitated by a sustained endotheliopathy and upregulation of endothelial surface adhesion molecules such as E-selectin, intracellular adhesion molecules (ICAM-1), and vascular adhesion molecule (VCAM-1). In normal conditions, the process has the physiologic purpose to destroy and dispose of injured tissue and debris, ultimately allowing astrocytes to produce microfilaments to synthesize scar tissue. However, with severe cerebral tissue disruption, this process becomes dysregulated and injurious to the host. There is a close relationship between the magnitude of post-traumatic leukocyte mobilization and the extent of brain edema and tissue loss in secondary brain injury (Fabricius, M. et al., Brain, 2006, 129:778-790).

It has been demonstrated that certain anticoagulants possess anti-inflammatory properties that can reduce LEU-mediated cerebral inflammation and swelling. In particular, unfractionated or low molecular weight heparins such as enoxaparin (ENX) blunt post-TBI leukocyte interactions with the BBB and improve neurological recovery independent of their anticoagulant properties (Nagata, K. et al., J. Trauma Acute Care Surg., 2016, 81:1088-1094; Li, S. et al. et al., J. Trauma Acute Care Surg., 2015, 79:78-84). Antithrombin-III (AT-III), another anticoagulant plasma protein, protects against endothelial injury in animal models of sepsis, ischemia-reperfusion, and acute lung injury (Maeda, A. et al., Pediatr. Int., 2011, 53:747-753; Rehberg, S. Crit. Care. Med., 2013, 41:439-446; Iba, T. et al., Thromb. Res., 2018, 171:1-6; Mizutani, A. et al., Blood, 2003, 101:3029-3036). Such effects are attributed to AT-III's ability to reduce peri-injury neutrophil accumulation and local vascular permeability in a fashion similar to that of enoxaparin (Uchiba, M. et al., Thromb. Res., 1998, 89:233-241).

AT-III is a plasma glycoprotein synthesized in the liver and belonging to the family of serine protease inhibitors (serpins). It is the predominant naturally occurring inhibitor of coagulation which mainly, but not exclusively, targets activated factor II (thrombin) and factor Xa. The clinical use of AT-III is currently limited to hereditary or acquired AT-III deficiency which is associated with inadequate anticoagulation and increased risk for thromboembolic events. Independent of AT-III's anticoagulation activity, various studies have demonstrated evidence of potent anti-inflammatory effects, particularly when AT-III plasma activity ranges from 150% to 200% of normal plasma levels (Uchiba, M. et al., Thromb. Res., 1998, 89:233-241; Uchiba, M. I. et al., Am. J. Physiol., 1996, 270:L921-930; Wiedermann, C. J. et al., Acta Med. Austriaca, 2002, 29:89-92). These anti-inflammatory effects appear to be mediated through interactions between AT-III and endothelium (Hoffmann, J. N. et al., Crit. Care Med., 2002, 30:218-225), producing a profound increase in endothelial prostacyclin production (Yamauchi, T. et al., Biochem. Biophys. Res. Commun., 1989, 163:1404-1411), which blocks PMN adhesion to ECs. AT-III has been shown to inhibit leukocyte rolling and adhesion to EC in a feline intestinal mesentery ischemia-reperfusion model (Ostrovsky, L. et al., Circulation, 1997, 96:2302-2310). In a similar model, Neviere and colleagues used small intestine intravital microscopy to demonstrate blunted LEU/EU adhesion by AT-III and reduced intestinal injury after endotoxemia (Neviere, R. et al., Shock, 2001, 15:220-225). The authors additionally showed how the addition of indomethacin, a prostacyclin inhibitor, reversed the blockade of LEU/EU interactions observed with AT-III (Hoffmann, J. N. et al., Am. J. Physiol., 2000, 279:C98-107). AT-III also binds to other surface leukocyte receptors (i.e. syndecan-4) on neutrophils, monocytes, and lymphocytes, directly inhibiting their interaction with endothelial cells (Mizutani, A. et al., Blood, 2003, 101:3029-3026; Honie, S. et al., Thromb. Res., 1990, 59:895-904). Therefore, while not wishing to be limited by theory, it was hypothesized that AT-III administered after TBI would reduce LEU-mediated cerebrovascular inflammation and decrease microvascular permeability, thereby reducing secondary brain injury and improving neurological recovery.

Enoxaparin, a low molecular weight heparin, has been shown to reduce brain edema, stroke lesion volume, and neurological impairment, specifically blocking LEU/EC interactions in the BBB and reducing tissue edema while enhancing neurological recovery after TBI (Li, S. et al., J. Trauma Acute Care Surg., 2015, 79:78-84; Wahl, F. et al., J. Neurotrauma, 2000, 17:1055-1065). Studies of how the effects of enoxaparin could be mediated through HMGB1 inhibition have been previously performed (Li, S. et al., J. Trauma Acute Care Surg., 2015, 79:78-84). In the current experiments, both ENX and AT-III blunted in vivo LEU/EC interactions and peri-contusional LEU mobilization, but only AT-III reduced local microvascular permeability. ENX administration in the current study was at lower doses and wider intervals. Without being limited by theory, this may explain why ENX failed to also reduce in vivo permeability. This data suggests that, similar to heparins, early administration of AT-III after TBI may block leukocyte activation at the injured BBB and mitigate against worsening cerebral swelling and secondary brain injury. AT-III has been associated with reductions in tissue edema and microvascular permeability in other studies as well. For example, AT-III attenuated vascular leakage via inhibiting neutrophil activation has been studied in animal lung injury models, wherein these studies also proposed that this effect is mediated by HMGB1 inhibition. Relatedly, AT-III has been shown to reduce spinal cord edema and improve incomplete spinal cord injury recovery through the facilitation of the spinal cord-evoked potentials and motor function.

The current study is the first report of AT-III modulation of LEU/EC interactions in the cerebral microcirculation following traumatic brain injury. In agreement with previous reports, the study linked live reductions in peri-contusional LEU/EC interactions and reduced microvascular leakiness. Further work may include the following studies:

1) Studies of human TBI to confirm that AT-III also modulates LEU/EC in humans.

2) A dose-response curve and measurement of serum AT-III activity in both ENX and AT-III animals as well as untreated animals will provide additional information as to the dose and dosing frequency necessary to treat TBI. The present studies used one dose of 250 IU/kg of AT-III after injury based on other disease processes and the manufacturer's recommendations.

3) Usage of a larger sample size and/or a different scale to study TBI recovery. Despite observing less weight loss in injured animals treated with AT-III, their GNT at 24 and 48 hours tended to demonstrate a trend of improved scores but failed to reach significance. This could reflect the small sample size and/or the that the GNT scale is not the optimal clinical tool for TBI recovery as it was designed primarily for stroke models.

4) A longer observation of several days to weeks to study TBI recovery of untreated versus treated animals. Although the 48 hour observation period was necessary to conduct the terminal surgery of intravital microscopy, which needs to occur two days after injury to capture the optimal time of leukocyte mobilization to the penumbra, this time frame may be insufficient to manifest small differences between groups.

5) Histological assessment and grading of cerebral hemorrhage. AT-III is a natural anticoagulant which may have caused unobserved bleeding and affected the animal neurological recovery, ultimately cancelling any benefit on cerebral swelling and potentially explaining the lack of statistically significant GNT score improvement from AT-III treatment.

### CONCLUSIONS

The present disclosure demonstrates the novel finding that early, post-TBI administration of AT-III reduces in vivo penumbral recruitment of LEUs and local microvascular permeability, resulting in greater animal weight loss recovery. Additional exploration of AT-III safety and efficacy in brain injured animals and humans will be performed in order to determine which preparations of AT-III can improve outcomes after TBI.

### Example 2

### Antithrombin III After TBI Improves Morris Water Maze Performance, Recovering Cued and Spatial Learning

### Methods

Experimental Design, TBI Model, and Study Groups

Experimental procedures were conducted with the approval of the University of Pennsylvania Institutional Animal Care and Use Committee. CD1 adult male mice (25-30 g) (Charles River Laboratories, Wilmington, Mass.) were acclimatized in standard housing with water and chow ad libitum for 7 days before experiments after which they underwent sham craniotomy or craniotomy accompanied by controlled cortical impact (CCI), a well-validated severe TBI rodent model (Pascual et al., Am J Surg. 2013;206(6):840-846; Smith et al., J Neurotrauma. 1995; 12(2):169-178). Mice were anesthetized with intraperitoneal ketamine (Hospira, Lake Forest, Ill.), xylazine (Akorn, Decatur, Ill.), and acepromazine (Boehringer Ingelheim, St. Joseph, Mo.) (100, 10, 1 mg/kg, respectively) followed by subcutaneous bupivacaine (0.5 mg/mL) for longer-term analgesia. After creation of a left-sided, 4-mm craniotomy between bregma and lambda sutures, the exposed left parietotemporal cortex was injured via a controlled cortical impactor (AMS201; AmScien Instruments, Richmond, Va.). Controlled cortical impact settings (3-mm-diameter impactor tip, impact velocity of 6 m/s, and cortical deformation depth of 1 mm) replicated severe TBI (Smith et al., J Neurotrauma. 1995; 12(2):169-178). Rodents were then randomly allocated to receive either: (1) intravenous (via femoral vein) pharmaceutical grade AT-III (250 IU/kg, donated by Grifols S. A., Durham, N.C.) or (2) an equal volume of normal saline IV (0.9% NS; Baxter; Deerfield, Ill.) as a vehicle (VEH). Doses were administered 0.5 and 24 hours after CCI (FIG. 1) as determined by previous published reports and the manufacturer's recommendations (ElSaadani et al., J Trauma Acute Care Surg. 2021; 90(2):274-280; Uchiba et al., Thromb Res. 1998; 89(5):233-241; Uchiba et al., Semin Thromb Hemost. 1997; 23(6):583-590).

Fifteen mice (ElSaadani et al., J Trauma Acute Care Surg. 2021; 90(2):274-280) were randomized into three groups: (1) sham craniotomy (no CCI) plus VEH (SHAM, n=5), (2) TBI and VEH (CCI-VEH, n=5), and (3) TBI and AT-III (CCI−AT-III, n=5).

### Morris Water Maze Exercises

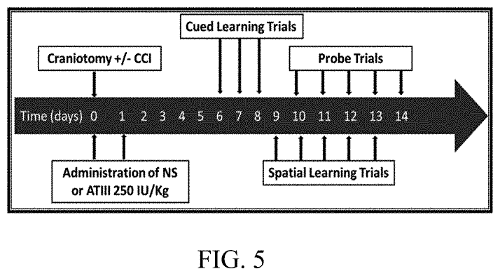

Learning and memory were evaluated in a Morris water maze (MWM) using a previously published protocol (Vorhees et al., Nat Protoc. 2006; 1(2):848-858; Jacovides et al., J Trauma Acute Care Surg. 2019; 87(3):552-558). After a recovery period of 6 days, each animal underwent daily. MWM trials of learning and memory for 9 consecutive days. The apparatus consisted of a black circular pool (diameter, 100 cm; height, 50 cm) filled with 21° C. water and fashioned with a black cylindrical platform (diameter, 10 cm; height. 23.5 cm) differentially submerged below or at the water surface. Experiments consisted of three types of swimming trials to assess differential learning and memory: (1) cued learning trials (days 6-8 post-TBI), (2) spatial learning trials (days 9-13 post-TBI), and (3) probe memory trials (days 110-14 post-TBI) (FIG. 5). All swimming trials were conducted and scored by the same operator, at the same time of day on all experiment days incorporating a computerized video-tracking and recording system over the pool to facilitate analysis (Ethovision; Noldus, Leesburg, Va.). Mice received a 10-minute rest period between trials, passively being warmed by a heating lamp.

### Cued Learning Trials

Four daily cued trials on days 6 to 8 post-TBI introduced animals to the water and pool. Cued trials served to reduce stress and to establish the MWM goal (navigation to the platform). The exposed platform was clearly visible to the swimming animal, adorned with a colorful marker and placed in one of four pool quadrants (southwest, southeast, northwest, northeast) randomly changed every day with no other wall visual cues provided. Animals were placed in the pool, in 1 of 4 randomly chosen locations relative to platform location. The video-tracking system collected performance parameters including time to reach the platform or the region around the platform (a predefined circular area around the platform), average swimming velocity, and distance and duration of swimming in each region. Animals were allowed 60 seconds to locate and then 15 seconds to stand on the platform before pool removal and passive warming. Animals that did not reach the platform within 60 seconds were gently guided to the platform and allowed to remain on it for 15 seconds before removal from the pool.

### Spatial Learning Trials

On postcraniotomy days 9 to 13, daily spatial learning trials evaluated the animals' ability to use environmental visual cues to locate and reach the platform. The MWM configuration was similar to that of the cued trials except that the platform remained in a fixed location (northwest) but hidden out of view as it was now submerged, although animals reaching the platform could stand on it and not be submerged. Colored pictograms were mounted on the MWM walls (north, south, east, and west) for animals to use for localizing of the submerged platform. Animals were again placed in the water facing outward from various points in relation to the platform so that they could see the wall pictograms but not the platform before entering the maze. Sixty seconds of swimming time and 15 seconds on the platform were again allowed before rest under the warming lamp. The same parameters as in the cued learning trials were collected using the video-tracking system.

### Probe Trials (Long-Term Memory)

On postcraniotomy days 10 to 14, daily probe trials were conducted immediately preceding spatial learning trials if both trials were conducted on the same day. Probe trials assessed long-term memory in navigating to the platform's prior location hinging on incorporating the prior days' visual cues into memory. During 30-second intervals, animals were allowed to freely swim in the pool now devoid of a platform. The duration, frequency, and time to first reach the area where the platform had been previously located during spatial trials were recorded to assess animals' cognitive recovery related to memory.

### Statistical Analysis

All data are presented as mean±SEM. Statistical analyses were performed using SPSS (SPSS, Chicago, Ill.; 2019). Differences between group means were compared using the Kruskal-Wallis test; significance was assumed for p<0.05.

### Results

Cued Learning Trials

In the cued learning trials, swimming velocity was similar in CCI−VEH and CCI−AT-III groups (26.5±1.2 cm/s and 28.4±1.1 cm/s, respectively); SHAM animals swam more rapidly (30.5±2.3, p<0.05 vs. CCI−VEH). The overall swimming distance was similar in both injured groups confirming the persistent sensorimotor effects from the TBI injury model.

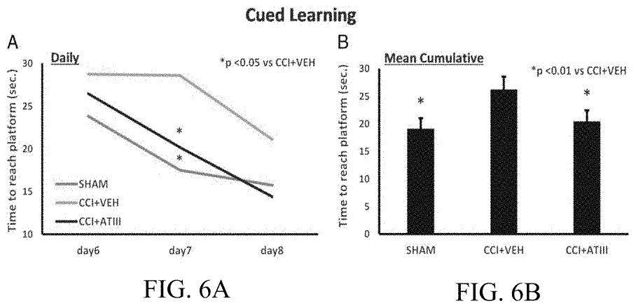

Learning patterns also differed between groups. Similar to SHAM, CCI−AT-III time to reach the exposed platform was less than the time for CCH−VEH counterparts (FIG. 6A). Controlled cortical impact-AT-III animals began cued trials outperforming CCI−VEH and eventually reached SHAM times by day 7. Controlled cortical impact-VEH animals only began to improve time-to-platform performance at the time that CCI−AT-III had demonstrated performance similar to SHAM (day 8). Relatedly, mean time to reach the platform was greatest in CCI−VEH (28.6±4.5 seconds). This time was nearly 30% greater than for either CCI−AT-III (20.1±3.1 seconds, p<0.05 vs. CCI−VEH) or SHAM (17.5±3.5 seconds, p<0.05 vs. CCI−VEH) groups. By the end of cued learning trials, cumulative cued learning (overall mean group time to reach the platform throughout days 6 to 8) was worst in CCI−VEH (26.1±2.4 seconds) compared with both CCI−AT-III (20.3±2.1 seconds, p<0.01) and SHAM (19.0±2.0 seconds, p<0.01), without significant differences between the latter two groups (FIG. 6B).

Spatial Learning Trials

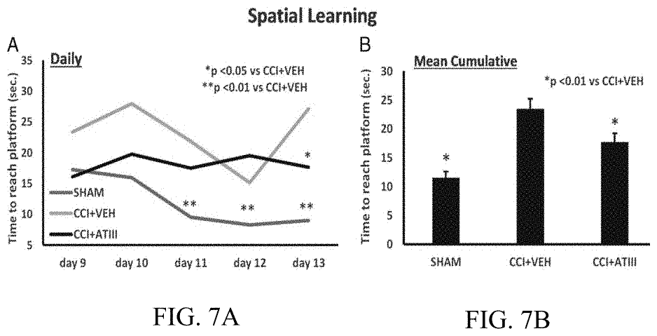

In the spatial learning trials occurring on postcraniotomy days 9 to 13, all groups now demonstrated similar swimming velocities. However, overall swim distance was greater in CCI−VEH (313.0±25.7 cm) and CCI−AT-III (306.9±27.9 cm) compared with SHAM (165.7±18.2 cm, p<0.01 vs. both CCI groups). Controlled cortical impact-VEH animals performed worst and demonstrated the slowest improvement. On day 13 (the last day of spatial learning trials), CCI−VEH swam longer (27.1±3.8 seconds) than CCI−AT-III (17.6±3.9 seconds, p<0.05) and SHAM (8.9±1.6 seconds, p<0.01 vs. CCI−VEH; FIG. 7A). Over the course of all 5 days of spatial learning trials, mean group cumulative swimming time was longest in CCI−VEH (23.4±1.8 seconds) compared with both CCI−AT-III (17.6±1.5 seconds, p<0.01) and SHAM (11.4±1.1 seconds, p<0.01) (FIG. 7B).

Probe Trials

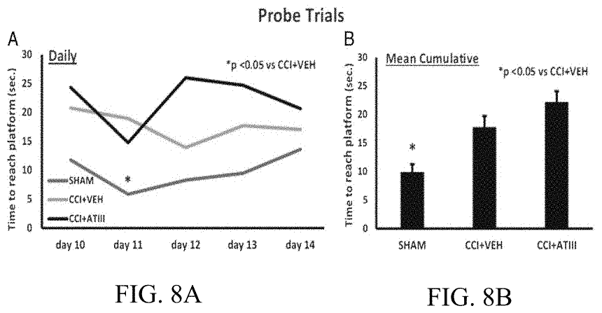

Probe trials disclosed no differences between the two injured groups in swimming velocity or distance, nor time to reach platform's prior location (FIGS. 8A-8B). Uninjured animals consistently fared better and demonstrated faster times to reach the platform's prior location.

Selected Discussion

This disclosure demonstrates that post-TBI AT-III improves certain aspects of cognitive recovery, mainly cued and spatial learning, but not memory in a murine model of blunt brain injury.

Despite decades of clinical and basic science research initiatives, there is, as of yet, no therapy that is routinely used to improve cognitive outcomes following TBI, especially with respect to recovery of cognitive impairment. Therefore, this model and its underpinning mechanism illuminate a potential pathway to support enhanced cognitive performance following blunt TBI. Moreover, since AT-III has a clearly defined pharmacology and a newly appreciated interface with microvascular surfaces, its ability to impact outcomes after injury is appropriately framed by a burgeoning appreciation for endotheliopathy on either side of the blood-brain barrier (Wu et al., Shock. 2020; 53(5):575-584). Inflammation that arises as a result of the primary brain injury is a key event in understanding the patient's ultimate trajectory. That such inflammation serves to prime the injured cerebral tissues for secondary injuriants appears to be plausible. This is especially true as a permeable blood-brain barrier coupled with neutrophil influx drive cerebral edema and intracranial hypertension, accelerating the risk of herniation if not successfully addressed (Shlosberg et al., Nat Rev Neurol. 2010; 6(7):393-403). Lesser degrees of injury support survival but may do so at the risk of motor, sensory or cognitive compromise. It is in this patient population in particular, that early effective therapy may be singularly effective in reducing poor functional outcomes, including those related to the genesis of auto-antibodies (Needham et al., J Neuroimmunol. 2019; 332:112-125).

Early after TBI, the blood-brain barrier temporarily becomes more permeable, allowing extravasation of fluid and activated circulating leukocytes into the adjacent cerebral parenchyma with subsequent activation of resident microglia and astrocytes (Lukaszewicz et al., Curr Opin Anaesthesiol. 2011; 24(2):138-143; Lucas et al., Br J Pharmacol. 2006; 147(1):232-240; Kubes et al., Brain Pathol. 2006; 10(1):127-135). Disruption to the hippocampal and cortical regions alters memory recovery in both human and animal TBI studies (Paterno et al., Curr Neurol Neurosci Rep. 2017; 17(7):52). The hippocampus receives spatial and nonspatial information about the environment via projections from the medial and lateral entorhinal cortex (Carbonell et al., Acta Neuropathol. 1999; 98:396-406). Hippocampal injury would thus be anticipated to result in deficits of spatial, learning, and memory capabilities. The MWM reliably evaluates hippocampal-related spatial navigation and references memory abilities rendering it an ideal method to assess the role of pharmacologic therapies on potentially relevant long-term outcomes (Vorhees et al., Nat Protoc. 2006; 1(2):848-858; Brody et al., Exp Neurol. 2006; 197(2):330-340; Scheff et al., J Neurotrauma. 1997; 14(9):615-627).

Controlled cortical impact is one of the most frequently used TBI models to study both early and late cognitive impairment after brain injury. A CCI injury model demonstrated a directly proportional relationship between the severity of the injury and MWM performance at 1 to 2 weeks post-TBI.31 Both learning and memory failure were noted regardless of injury severity and irrespective of the absence of grossly identifiable tissue damage. Local injury may lead to tissue damage or death but, more importantly, can lead to integrative deficits in more remote domains as well. It was previously demonstrated how CCI initiates specific neuroinflammatory changes that are immunohistochemically visible in the penumbral area of the injury and the hippocampus (Suto et al., J Trauma Acute Care Surg. 2018; 85(2):275-284), cementing the relationship of injury remote from the site of impact. These observations underscore the need for a systemic approach to blunt TBI that complements local measures that may include hematoma evacuation as appropriate.

Antithrombin III is a plasma glycoprotein synthesized in the liver and is the primary physiological inhibitor of thrombin and other serine proteases of the coagulation cascade. While the approved clinical use is limited to treating AT-III deficiency-related clotting, AT-III exerts potent anti-inflammatory effects (Allingstrup et al., Cochrane Database Syst Rev. 2016; 2(2):CD005370; Wiedermann et al., Acta Med Austriaca. 2002; 29(3):89-92; Uchiba et al., Am J Physiol. 1996; 270(6):L921-L930). Inflammation reduction occurs most prominently at twice the normal plasma concentration of AT-III, appears to be endothelially mediated, and is related to augmented prostacyclin production (Hoffmann et al., Crit Care Med. 2002; 30(1):218-225). Reduced reactive oxygen species and neutrophil-derived protease release are two described methods of inflammation reduction (Hoffmann et al., Crit Care Med. 2002; 30(1):218-225; Nevière et al., Shock. 2001; 15(3): 220-225). Antithrombin III has been demonstrated to improve outcome in a number of tissue injury models, not just following blunt TBI as in the current study. In a rodent sepsis model, AT-III reduced both organ failure and mortality (Iba et al., Intensive Care Med. 2005; 31(8):1101-1108). Using intravital microscopy of intestinal venules, AT-III similarly reduced microvascular neutrophil rolling and endothelial adhesion in a feline ischemia-reperfusion injury model (Ostrovsky et al., Circulation. 1997; 96(7):2302-2310). To wit, AT-III also reduces endothelial leakage, microvascular permeability, and tissue edema by inhibiting neutrophil activation—observations that have been previously linked to high-mobility group box 1 inhibition in acute lung injury and sepsis (Rehberg et al., Crit Care Med. 2013; 41(12):e439-e446; Hagiwara et al., Intensive Care Med. 2008; 34(2):361-367). Incomplete spinal cord injury recovery may also benefit from AT-III as assessed by earlier recovery of spinal cord-evoked potentials and motor function in a rodent model (Arai et al., Spine. 2004; 29(4):405-412). Finally, in the central nervous system, it was recently demonstrated that early post-TBI administration of AT-III reduces in vivo penumbral leucocyte recruitment and blunts local microvascular permeability, subsequently decreasing brain edema and resulting in greater animal weight loss recovery after blunt injury (ElSaadani et al., J Trauma Acute Care Surg. 2021; 90(2):274-280). Indeed, the correlation between postinjury tissue swelling and poor outcome is nowhere more relevant than in TBI where cerebral swelling can rapidly result in death through brain herniation to accommodate the increased intracranial tissue volume. Since these observations now cross species and models, they perhaps suggest a more ubiquitous mechanism and role for AT-III after tissue injury in support of organ recovery as a key step toward functional outcome enhancement. The results of the present study demonstrate the durability of the beneficial effects of AT-III on cognitive recovery following TBI, as this inquiry spans weeks, and not the more common assessment measured in hours or a few days.

### CONCLUSIONS

Early post-TBI administration of AT-III improves neurocognitive recovery weeks after injury. This improvement appears specifically related to recovering learning abilities but not long-term memory. Additional exploration of AT-III's safety and efficacy is warranted to determine if AT-III can also enhance human cognitive recovery after blunt TBI. Given AT-III's anti-inflammatory properties, it is poised to be a powerful tool that augments the armamentarium of neurocritical care management in the care of injured patients.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the disclosure. The appended claims are intended to be construed to include all such embodiments and equivalent variations.

### Enumerated Embodiments

Embodiment 1 provides a method of treating a traumatic brain injury (TBI) in a subject, the method comprising: administering to the subject a composition comprising a therapeutically effective amount of antithrombin-III (AT-III).

Embodiment 2 provides a method of embodiment 1, wherein the composition further comprises a low molecular weight heparin.

Embodiment 3 provides the method of embodiment 2, wherein the low molecular weight heparin is enoxaparin.

Embodiment 4 provides the method of any one of embodiments 1-3, wherein the composition is administered to the subject intravenously or subcutaneously.

Embodiment 5 provides the method of any one of embodiments 1-4, wherein the method of treating a TBI in the subject reduces secondary injury in the subject following the TBI by blocking neurovascular inflammation, reducing cerebral endothelial-leukocyte interactions, decreasing blood brain barrier permeability, or a combination thereof.

Embodiment 6 provides the method of any one of embodiments 1-5, wherein the method of treating a TBI in the subject improves post-TBI cognitive recovery in the subject.

Embodiment 7 provides the method of any one of embodiments 1-6, wherein the method of treating a TBI in the subject improves post-TBI learning and memory in the subject.

Embodiment 8 provides the method of any one of embodiments 1-7, wherein the step of administering to the subject a composition comprising a therapeutically effective amount of AT-III comprises the step of administering the composition within 72 hours following the TBI.

Embodiment 9 provides the method of embodiment 8, wherein the step of administering to the subject a composition comprising a therapeutically effective amount of AT-III comprises the step of administering the composition within one hour following the TBI.

Embodiment 10 provides a composition for treating a traumatic brain injury (TBI) in a subject, the composition comprising a therapeutically effective amount of antithrombin-III (AT-III).

Embodiment 11 provides the composition of embodiment 10, wherein the composition further comprises a low molecular weight heparin.

Embodiment 12 provides the composition of embodiment 10, wherein the low molecular weight heparin is enoxaparin.

Embodiment 13 provides the composition of any one of embodiments 10-12, wherein the composition is administered to the subject intravenously or subcutaneously.

Embodiment 14 provides the composition of any one of embodiments 10-13, wherein the composition reduces secondary injury in the subject following the TBI by blocking neurovascular inflammation, reducing cerebral endothelial-leukocyte interactions, decreasing blood brain barrier permeability, or a combination thereof.

Embodiment 15 provides the composition of any one of embodiments 10-14, wherein the composition improves post-TBI cognitive recovery in the subject.

Embodiment 16 provides the composition of any one of embodiments 10-15, wherein the composition improves post-TBI learning and memory in the subject.

Embodiment 17 provides the composition of any one of embodiments 10-16, wherein the composition is administered to the subject with TBI within 72 hours following the TBI.

Embodiment 18 provides the composition of embodiment 17, wherein the composition is administered to the subject with TBI within one hour following the TBI.

Embodiment 19 provides the composition of any one of embodiments 10-15, wherein the composition further comprises a pharmaceutically acceptable carrier.

What is claimed is:

1. A method of treating a traumatic brain injury (TBI) in a subject, the method comprising: administering to the subject a composition comprising a therapeutically effective amount of antithrombin-III (AT-III).

2. The method of claim 1, wherein the composition further comprises a low molecular weight heparin.

3. The method of claim 2, wherein the low molecular weight heparin is enoxaparin.

4. The method of claim 1, wherein the composition is administered to the subject intravenously or subcutaneously.

5. The method of claim 1, wherein the method of treating a TBI in the subject reduces secondary injury in the subject following the TBI by blocking neurovascular inflammation, reducing cerebral endothelial-leukocyte interactions, decreasing blood brain barrier permeability, or a combination thereof.

6. The method of claim 1, wherein the method of treating a TBI in the subject improves post-TBI cognitive recovery in the subject.

7. The method of claim 1, wherein the method of treating a TBI in the subject improves post-TBI learning and memory in the subject.

8. The method of claim 1, wherein the composition is administered within 72 hours following the TBI.

9. The method of claim 8, wherein the composition is administered within one hour following the TBI.

10. A composition for treating a traumatic brain injury (TBI) in a subject, the composition comprising a therapeutically effective amount of antithrombin-III (AT-III).

11. The composition of claim 10, wherein the composition further comprises a low molecular weight heparin.

12. The composition of claim 11, wherein the low molecular weight heparin is enoxaparin.

13. The composition of claim 10, wherein the composition is administered to the subject intravenously or subcutaneously.

14. The composition of claim 10, wherein the composition reduces secondary injury in the subject following the TBI by blocking neurovascular inflammation, reducing cerebral endothelial-leukocyte interactions, decreasing blood brain barrier permeability, or a combination thereof.

15. The composition of claim 10, wherein the composition improves post-TBI cognitive recovery in the subject.

16. The composition of claim 10, wherein the composition improves post-TBI learning and memory in the subject.

17. The composition of claim 10, wherein the composition is administered to the subject with TBI within 72 hours following the TBI.

18. The composition of claim 17, wherein the composition is administered to the subject with TBI within one hour following the TBI.

19. The composition of claim 10, wherein the composition further comprises a pharmaceutically acceptable carrier.
